(19) United States
(12) Patent Application Publication
(10) Pub. No.: US 2005/0209649 A1
(43) Pub. Date: Sep. 22, 2005

(54) Mechanical sensing system for cardiac pacing and/or for cardiac resynchronization therapy

(76) Inventor: Bozidar Ferek-petric, Zagreb (HR)

Correspondence Address: MEDTRONIC, INC., 710 MEDTRONIC PARKWAY NE, MS-LC340, MINNEAPOLIS, MN, 55432-5604, US

(21) Appl. No.: 10/802,953

(22) Filed: Mar. 17, 2004

(51) Int. Cl. A61N 1/365

(52) U.S. Cl. 607/17

(57) ABSTRACT

According to the present invention a sensing means is provided for chronically measuring and/or sensing contractions of a right ventricle (RV) and/or a left ventricle (LV). The sensing means can include a tensiometric sensor, a single or a multiple axis accelerometer to measure peak endocardial acceleration due to atrial and ventricular depolarizations. For example, a tensiometric stylet, disposed within a portion of the coronary sinus, great vein, or branches of the great vein, simultaneously senses atrial contractions, RV contractions and LV contractions and provides an output signal related thereto. When an atrial contraction occurs, pacing stimulation is delivered to the LV upon expiration of a predetermined A-LV delay interval. The A-LV delay interval for the pacing therapy is adjusted so as to avoid delay between the respective contractions of RV and LV, respectively, and thereby promote ventricular synchrony.

### CROSS REFERENCE TO RELATED APPLICATIONS

This patent application relates to a co-pending non-provisional U.S. patent application by Hill, namely Ser. No. 10/000,474 (Atty. Dkt. P-8968.00) filed 26 Oct. 2001 and entitled, “System and Method for Bi-Ventricular Fusion-pacing;” a non-provisional U.S. patent application by Pilmeyer and van Gelder; namely Ser. No. 10/______ (Atty. Dkt. P-11417.00) filed 17 Mar. 2004, and entitled, “APPARATUS AND METHODS FOR ‘LEPARS’ INTERVAL-BASED FUSION-PACING;” and a non-provisional U.S. patent application by Burnes and Mullen entitled, “APPARATUS AND METHODS OF ATRIAL-BASED BI-VENTRICULAR FUSION PACING” filed as Ser. No. 10/______ (Atty. Dkt. P-11471.00) filed 17 Mar. 2004 and the entire contents of each is hereby incorporated by reference herein.

### FIELD OF THE INVENTION

The present invention relates to cardiac pacing systems. In particular, the invention relates to a cardiac pacing system utilizing one or more mechanical sensors that continuously provide output signals related to the timing and magnitude of contractions during a cardiac cycle so that delivery of therapeutic pacing therapies, such as cardiac resynchronization therapy (CRT), can be optimized without masking, or “blanking,” a part of each cardiac cycle.

### BACKGROUND OF THE INVENTION

Prior art pacing systems typically employ at least one pair of pacing electrodes that alternately deliver pacing stimulus to at least one chamber of a heart and detect the resulting cardiac response. When the resulting cardiac response is detected by electrodes disposed in or about the heart, a temporal tracing of the cardiac activity is referred to as an electrogram (EGM). When the electrodes are disposed in the same chamber wherein pacing stimulation is delivered the EGM is oftentimes referred to as a “near-field” EGM. Following delivery of the pacing stimulus operative electronic sensing circuitry coupled to the electrodes are oftentimes switched off, or “blanked,” for a period of time. Such blanking simply blocks the pacing-level electrical stimulus from overwhelming the sensing circuitry and thus protects the automatic gain control (AGC) amplifiers oftentimes coupled to the sensing electrodes. Another reason for blanking cardiac pacing stimulus signals relates to the localized polarization currents generated at the tissue-electrode interface. Since such polarization currents do not reflect physiologic activity, the blanking interval typically eliminates electrical signals resulting therefrom.

Such blanking thus eliminates a portion of each cardiac cycle during delivery of pacing stimulus. The blanking also imposes limits that can reduce the opportunity to detect an arrthymia episode due to the fact that an arrthymia, such as a tachycardia episode or a premature contraction event, may begin or occur without detection. In addition, prior art pacing systems that rely upon sensed electrical activity such as that contained in a near-field EGM are inherently out of phase with the actual evoked or intrinsic mechanical activity of the heart. That is, the electrical depolarization and repolarization wavefronts precede the actual physical contraction and relaxation and recovery phases of the myocardium. Also, in certain circumstances an EGM waveform (or ECG waveform derived from surface-based electrodes) can appear normal while actual physiologic activity is lacking or non-existent. This phenomenon is often referred to as pulse-less electrical activity, also known as electromechanical dissociation (EMD), and refers to a condition wherein essentially no blood is ejected from the ventricles (i.e., essentially null stroke volume and cardiac output) but operative sensing circuitry or manual inspection of an EGM or ECG suggests relatively normal cardiac activity. In the event that EMD persists without relatively rapid intervention (e.g., cardio-pulmonary resuscitation, defibrillation or cardioversion therapy delivery and the like) death can result.

In U.S. Pat. No. 5,261,418 (the '418 patent) a tensiometric-type mechanical sensor for the cardiac contractions measurement is disclosed, illustrated and claimed. The contents of the '418 patent are hereby incorporated by reference. In the '418 patent the following statements and advantages of the invention claimed therein appear. According to the '418 patent, a system for myocardial tensiometry is incorporated within an implantable electrotherapy apparatus to measure mechanical contractions of heart muscle. The tensiometric system of the '418 patent is preferably formed by an elastic strip made of either piezoelectric material or resistive material and mechanical stresses imparted to the strip produces either an electric voltage or a varies electrical impedance thereof, respectively. Thus, the '418 provided a device having an elastic tensiometric strip adapted to be mechanically coupled to heart muscle.

The invention disclosed in the '418 patent provides a device with the capability of either analyzing electric signals or measuring variations in the electrical impedance produced within the tensiometric strip caused by cardiac muscle contractions.

As recited in the '418 patent, the invention relates to providing a device capable of monitoring mechanical activity of a heart in order to check whether pacing therapy pulses are followed by a mechanical contraction (i.e., said pulses have “captured” a chamber). Furthermore, the '418 patent provides detection of mechanical movements of a heart which are characteristic for certain cardiac rhythms, thus enabling detection of certain pathologic cardiac rhythms. The '418 patent described a sensor that indicates physical stress related to the tensiometric measurement of acceleration. The '418 patent posited that significant differences remain between measurement of acceleration of a portion of a lead and tension forces impinging upon the lead. As is well known, the output signal of an accelerometer is a function of the first derivative of the velocity of the lead (displacement from a first position to a second position). A vector describing this velocity is understood to be oriented orthogonal to the longitudinal axis of the lead body. This is due, at least in part, to the fact that the implanted lead, especially following implantation within a heart by means of the ingrowth of fibrotic tissue (and/or a so-called active fixation mechanism such as a helix or hook-shaped member). The inventor believes that such ingrowth inhibits radial movement of the lead body relative to adjacent myocardial tissue. The signal from an accelerometer can be viewed as primarily influenced by two components: linear (largely radial) intracardiac acceleration forces upon a lead—caused by cardiac contractions—and multidirectional acceleration forces caused by movements of the entire human body and/or movement of vehicles used transport a body (e.g., due to a vehicle traversing terrain or fluid). Therefore, accelerometer signals may oftentimes over-sense acceleration forces influencing a human body which then impedes accuracy, specificity and sensitivity of an output signal from an accelerometer sensor. Sensitivity rises when the lead is enclosed within a fibrotic channel (or encapsulated region) that significantly attenuates the radial component of an output signal from an accelerometer due to the forces generated during cardiac contractions. Furthermore, radial acceleration of the lead can be detrimentally influenced by intracardiac blood that attenuates direct energy transfer from the myocardium to the accelerometer. Assuming a lead implanted in the middle of the intracardiac cavity (providing free radial movement of the sensor within the right ventricle), cardiac contraction energy transfer to the lead occurs primarily at the tip of the lead. Therefore, the elastic lead body also serves to attenuate energy transfer between the accelerometer and the lead tip (i.e., and the myocardium).

Contrary to known systems, in the system disclosed in the '418 patent the cardiac contraction energy is transformed directly into mechanical (stretching) energy within a transducer. Thus, the transducer measures and, optionally, processes the signal produced by the sensor within the transducer, which mechanically couples to the myocardium. Accordingly, each cardiac contraction provides a signal having amplitude and frequency characteristics representing the same characteristics as the contraction itself, consequently enabling signal processing in such a way as to obtain information about the contraction amplitude and velocity as parameters for cardiac electrotherapy control. Thus, no external mechanical energy can impede the tensiometry signal and, in addition, fibrotic tissue ingrowth there is no significant influence of any fibrotic tissue on the signal.

In addition to the foregoing issued U.S. patent, published international patent application PCT/EP 95/00113 (published on 20 Jul. 1995) entitled, “Cardiac Electrotherapy System with Cardiac Contraction Sequence Measurement,” (as WIPO publication number WO95/19201) provides additional context for the present invention. This publication describes a system that measures the timing between the contractions of various cardiac chambers for the purpose of cardiac arrhythmia detection and classification. Furthermore, studies presented at NASPE Washington 2000 emphasized the importance of the cardiac contraction sequence. Other researchers have shown that peak endocardial acceleration serves as a valid parameter for measuring improvement of cardiac resynchronization. Likewise, according to still other cardiac researchers, left ventricular (LV) contraction asynchrony may be the predictor for cardiac arrhythmia risk particularly in heart failure (HF) patients suffering from dilated cardiomyopathy. Moreover, still other cardiac researchers have disclosed a method wherein AV delay optimization is employed to achieve maximum peak endocardial acceleration.

However, the inventor suggests that to date no other cardiac research personnel or inventor has invented an effective mechanical sensing apparatus and methods of utilizing same to optimize CRT delivery.

### SUMMARY OF THE INVENTION

According to the present invention a sensing means is provided for measuring and/or sensing contractions of the right ventricle (RV) and the left ventricle (LV). The sensing means may comprise a tensiometric sensor (such as that disclosed in the '418 patent) or a single (or multiple) axis accelerometer adapted to measure peak endocardial acceleration.

In one embodiment of the invention, a tensiometric stylet portion of the sensing means is deployed through the coronary sinus into a portion of the great vein, or braches thereof, to sense atrial contractions, RV contractions and LV contractions. Upon sensing an atrial contraction, the LV pacing therapy stimulation is delivered upon expiration of a predetermined interval (i.e., an A-LV delay). The A-LV delay interval (for LV pacing therapy) is adjusted in such a way as to avoid delay between the respective contractions of RV and LV, respectively during delivery of a form of cardiac resynchronization therapy (CRT).

The disclosure provides methods and structures for monitoring cardiac contractions using a single sensor during pacing therapy delivery that offer significant advances over the prior art. Such a sensor coupled to a medical electrical lead can be strategically deployed to effect mechanical communication with a single portion of myocardium and thereby detect all atrial and ventricular contractions. Continuous detection (without any interruption or delay as is common with prior art techniques) allows optimal sensing of cardiac activity. Using output signals from the mechanical sensor enables optimization of a variety of cardiac pacing modalities. For example, such sensor output signals may be used to adjust timing of pacing stimulus during bi-ventricular CRT delivery, single-stimulus (so-called “fusion-based”) pacing therapy delivery, and extra-systolic stimulation therapy delivery, among others.

Additionally, according to the invention at least one medical electrical lead is deployed into operative mechanical communication with the myocardium. Said lead having a mechanical sensor (e.g., accelerometer and/or tensiometric sensor) operatively coupled to sense relative contractile delay between the RV and the LV and provide an output signal thereof. Said output signal can be advantageously utilized for the purposes of optimizing A-V delay for LV pacing, V-V delay for CRT delivery, capture detection of a cardiac chamber, and the like.

### BRIEF DESCRIPTION OF THE DRAWINGS

The drawings are not drawn to scale and, as applicable, like elements are numbered the same; in addition, those of skill in the art will appreciate that the drawings are illustrative and not exhaustive of the aspects and variety of embodiments of the present invention.

FIG. 1 depicts a pair of temporal traces, an upper trace illustrating PQRST cardiac complexes wherein the ventricles are not synchronously depolarizing and a lower trace depicting a mechanical tensiometric sensor output signal corresponding to the cardiac complexes depicted in the upper trace.

FIG. 2 depicts a pair of temporal traces, an upper trace illustrating PQRST cardiac complexes and a lower trace depicting a mechanical tensiometric sensor output signal corresponding to the upper trace, during iterative adjustment of atrio-ventricular (A-V) intervals according to the present invention.

FIG. 3 depicts a flow chart illustrating one algorithm for performing the A-V interval adjustment(s) according to the present invention.

FIG. 4 depicts an elevational side view in cross section of a prior art curvilinear tensiometric-type mechanical sensor adapted for transvenous delivery.

FIG. 5. depicts an elevational side view in cross section of a substantially linear prior art tensiometric-type mechanical sensor adapted for transvenous delivery.

FIG. 6 is an illustration of transmission of the cardiac depolarization waves through the heart in a normal intrinsic electrical activation sequence.

FIG. 7 is a schematic diagram depicting a three channel, atrial and bi-ventricular, pacing system for implementing the present invention.

FIG. 8 is a simplified block diagram of one embodiment of IPG circuitry and associated leads employed in the system of FIG. 7 for providing three sensing channels and corresponding pacing channels that selectively functions in an energy efficient ventricular-fusion pacing mode according to the present invention.

### DETAILED DESCRIPTION OF THE ILLUSTRATED EMBODIMENTS

According to the present invention, a system and methods of delivering cardiac resynchronization therapy (CRT) and other pacing therapies is provided wherein a mechanical sensor signal, free of blanking intervals typically imposed on electrical cardiac sensing circuitry, is used to rapidly and accurately tune atrio-ventricular (A-V) and/or interventricular (V-V) cardiac pacing intervals. In addition, the apparatus can be employed to distinguish between capture and loss of capture (LOC) of one or more cardiac chambers during pacing therapy delivery.

In one embodiment, the system includes a tensiometric sensor (coupled to a cardiac pacing lead or a stylet) or an accelerometer sensor mechanically coupled to both the RV and the LV (e.g., disposed in a portion of the coronary sinus, great vein or branches of the great vein). In another embodiment, the system comprises only tensiometric stylet within the coronary sinus, great vein or branches of the great vein.

In other embodiments, a distal portion of a pacing lead is disposed in a portion of the great cardiac vein and coronary sinus and comprises a tension-type sensor such as the tensiometric stylet. The tensiometric stylet yields three signals: a first signal corresponding to atrial contraction events (RA and LA), a second signal corresponding to RV contraction events, and a third signal corresponding to LV contraction events. A time interval between the second and the third signal corresponds to an interventricular contraction delay.

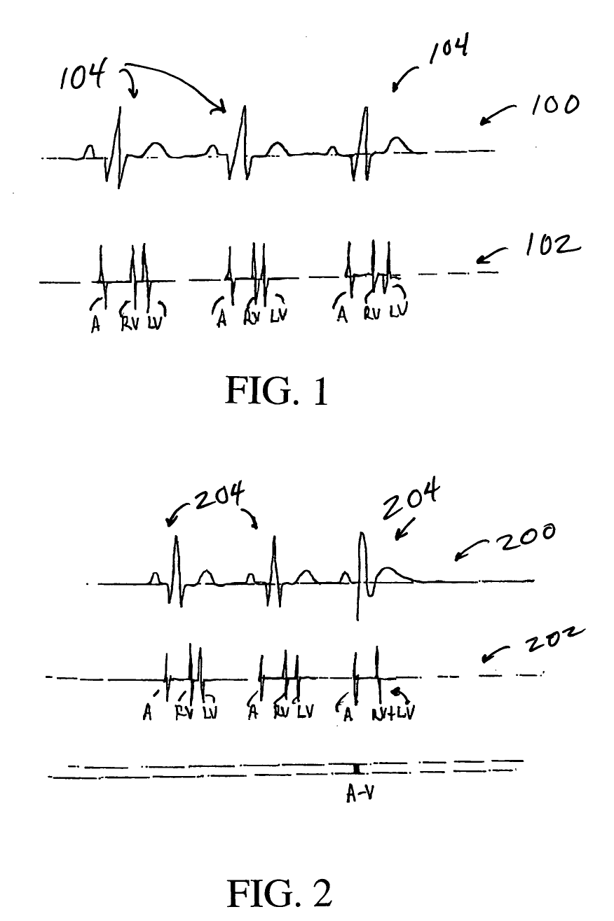

Referring now to FIG. 1, which depicts a pair of representative temporal traces 100,102; namely, electrically-sensed cardiac activity 100 and a corresponding output signal 102 from a tensiometric tranducer. The upper trace 100 illustrates the familiar morphology of several electrical PQRST cardiac complexes 104 comprising sensed depolarization and repolarization wavefronts. The lower trace 102 depicts an output signal from a tensiometric sensor mechanically coupled to a heart to detect the physical contractions corresponding to the cardiac complexes 104 of upper trace 100. The deflections of lower trace 102 are labeled with an “A” for atrial contraction events, “RV” for right ventricular contraction events, and “LV” for left ventricular contraction events.

For convenience, in FIG. 1 the cardiac complexes 104 are depicted as temporally aligned with the output signal 102 from the tensiometric transducer. However, as understood by those of skill in the art, the electrical (or ionic) activity sensed with electrodes operatively coupled to the myocardium actually precedes the resulting mechanical response as the cardiac myocytes contract and relax. In operation, assuming relatively immediate transfer of mechanical motion to an output signal from the tensiometric transducer, the present invention offers the advantage of enabling essentially real-time control of a cardiac pacing system. That is, output signals from cardiac activity sensed via one or more tensiometric transducers, as set forth herein, inherently include the collective impact of all the dynamic physiologic (and non-physiologic) characteristics of a patient's heart. For example, nodal and/or conduction anomalies or defects, presence of acute ishemic events, myocardial infarcts, ectopic foci and re-entrant pathways, and the like.

Continuing with FIG. 1, inspection of the lower trace 102 of FIG. 1 reveals that the sensed contractile activity of the LV and RV are not synchronously occurring as shown by the three discrete mechanical deflections (A, RV, LV) of the lower trace 102. As mentioned above, the deflections of lower trace 102 (i.e., the signals corresponding to the mechanical deflections for each cardiac cycle) correlate to the timing of the PQRST complexes 104 depicted in FIG. 1 but should actually precede, in time, the complexes 104.

According to one embodiment of the invention, a stylet of cardiac lead having a tensiometric sensor coupled to a distal portion thereof, is deployed through the ostium of the coronary sinus through a portion of the great cardiac vein and/or branches thereof to a suitable location near the atria and LV. As a result, when the atria contract the entire region around the coronary sinus and great vein moves superiorly. Thus, as a consequence of each contraction the tensiometric sensor coupled to the distal part of the stylet or cardiac lead moves (and/or bends) thereby yielding a mechanical signal. This signal appears to emanate from a proximal portion of the tensiometric transducer. In the case of contractions of the RV, the entire cardiac base shifts inferiorly consequently imparting mechanical motion to the tensiometric transducer disposed within a portion of the great vein. The signals produced as a result of RV contractions appear to also emanate from the proximal portion of the tensiometric transducer. In a similar manner, contractions of the LV also impart mechanical motion to the tensiometric transducer, although such signals appear to emanate from a distal portion of the transducer. Thus, as a result of an appropriately situated tensiometric transducer disposed at least partially within a portion of the great vein and/or portions thereof mechanical contractions of the cardiac chambers are readily sensed without delay as they occur. In contrast, as is known in the art of electrical cardiac pacing and sensing systems ionic transfer across the surface of the myocardium is detected via pairs of electrodes disposed on or about the heart. The signals detected by the electrodes represent depolarization and repolarization wavefronts that necessarily precede the actual systolic contractions and diastolic relaxations that circulate blood throughout the human body.

Referring now to FIG. 2, the familiar temporal representation 200 of electrical (or ionic) activity accompanying three cardiac cycles 204 is depicted. Of the three cycles, two depict ventricular asynchrony, as indicated by the atrial contraction followed by a pair of ventricular contractions as depicted in the lower trace of three sets of tensiometric sensor output signals 202. The other cardiac cycle 204 produced ventricular synchrony, as shown in the third set of tensiometric sensor output signal 202. In this output signal an atrial contraction resulted in the signal (labeled “A”) and the contraction of the RV and LV produced a single event (labeled “RA+LV”).

In one form of the present invention, instead of electrically sensing contractions of the atria, tensiometric sensing is implemented to control a variety of pacing modalities (e.g., sensing atrial activity to initiate the A-V delay for VDD pacing). In addition, especially for those patients who require only LV pacing with or without so-called fusion-pacing (but not bi-ventricular pacing), a single cardiac lead having a tensiometric sensor proximal of the distal end and at least one electrode at or near the distal end, can be used for chronic single-lead VDD pacing therapy delivery. According to general aspects of this form of the invention, a combination of one or more electrodes (e.g., tip, ring, cardioversion, defibrillation coils, etc.) and one or more tensiometric sensors are deployed into contact with the myocardium so that both mechanical and electrical cardiac performance can be monitored. In particular, such cardiac lead systems could be used to very efficiently monitor cardiac performance in a non-pacing “ODO” programmed pacemaker. Of course, “ODO” refers to a programmed pacemaker operating in a sensing-only mode (i.e., for each cardiac cycle: O=no pacing, D=dual chamber sensing, O=no inhibit or trigger of pacing therapy for the subsequent cardiac cycle). Such a mode can be employed in the event that a patient's stable normal sinus rhythm (NSR) emerges or for periods of time when observing NSR (e.g., timing, magnitude, rate of cardiac activity). As noted previously, in the event that mechanically-based tensiometric sensor signals are compared to, combined with, or used to control electrically-based signals such as ECG, EGM, or cardiac pacing signals, the temporal offset between the signals should be considered (e.g., adjusted or synchronized). As a result, if an atrial contraction signal is obtained using a tensiometric sensor initiates an A-V interval being significantly shorter than conventional A-V interval for electrical P-wave sensing (whether evoked or intrinsic).

In order to achieve and maintain ventricular synchrony, the interval between the RV and LV contractions are monitored, stored and/or measured. In one form of the invention, an iterative process of applying different operating A-V intervals and monitoring RV and LV contractions is performed until the RV and the LV tensiometric sensor output signals align (i.e., can be temporally superimposed into one wave) in order to produce the simultaneous ventricular contraction whereby the RV is depolarized spontaneously while the LV is paced. That is, achieve LV-only pacing fusion depolarization.

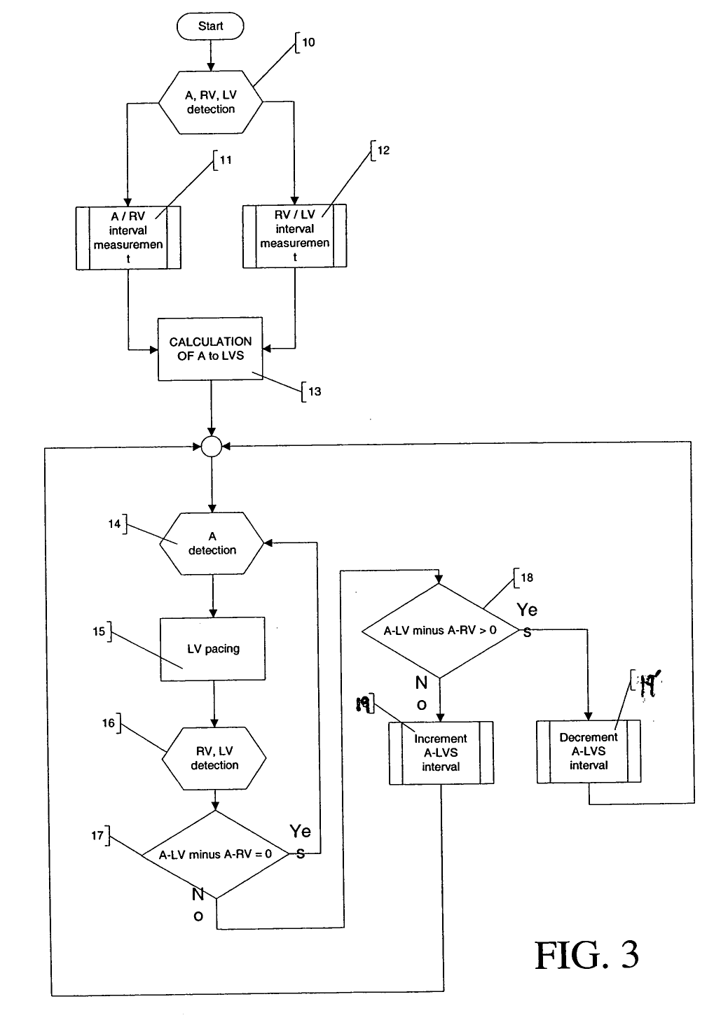

FIG. 3 depicts an algorithm for the A-V interval regulation using mechanical sensing techniques for LV-only pacing therapy delivery. The depicted process begins at step 10, whereby atrial, RV and LV contractions are detected. Then at steps 11,12 the A-RV interval is determined (i.e., the period of time between atrial and RV contraction) and the V-V interval (i.e., the period of time between RV and LV contractions). At step 13, in order to compensate for a relative delay of the LV contraction, the atrial-to-LV stimulus (A-LVS) interval for LV-only pacing is calculated. The A-LVS calculation can be viewed as a simple calculation wherein inter-ventricular contraction delay, or “time” (IVCT), is deducted from the total A-LV contraction delay. Then, at step 14 a subsequent atrial contraction is again sensed, and at step 15 the left pacing stimulus (LVS) issued. The V-V (LV-RV) interval is again measured at step 16 and compared at step 17 to determine whether the RV and LV contraction events are simultaneous. If the RV and LV contract simultaneously, then the (VDD) pacing continues (steps 14 and 15).

However, if at step 17 the RV and LV contractions are determined not to be simultaneous (or within a range deemed essentially physiologically simultaneous), then step 18 is performed to determine whether the LV contraction proceeds or lags the RV contraction. If the LV contraction lags the RV contraction, the LV-only pacing A-V interval (A-LVS interval) is incremented at step 19. If the LV contraction precedes the RV contraction, the LV-only pacing A-V interval (A-LVS interval) is decremented at step 19′. The methods of the present invention may be practiced under micro-processor control by executing instructions stored on a computer-readable medium, as is well-known in the art.

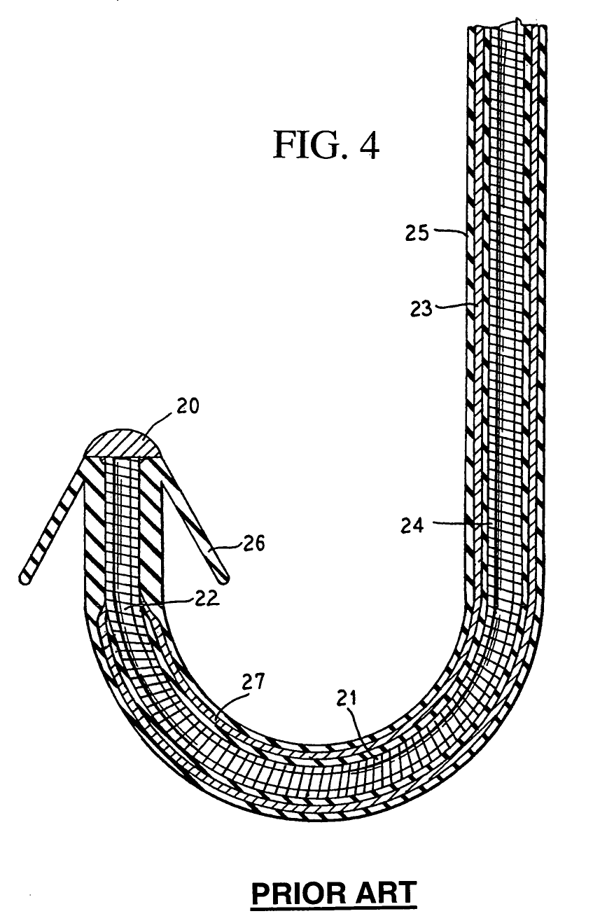

In the embodiment of FIG. 4, there is shown a distal part of a J-shaped unipolar pacing lead having an electrode 20 at the tip. The electrode 20 is electrically connected with the central pin of a connector (not shown) at the proximal part of the lead (not shown) by means of the lead conductor 21 having a stylet channel 22. The lead has another coaxial lead conductor 23 that is connected with the ring of the same connector (not shown). Two helically wounded lead conductors are isolated by means of an inner insulation 24 and an outer insulation 25. The surface of the outer insulation 25 may have some means for lead fixation at the tip of the lead. In the disclosed embodiment, tines 26 are shown only for example. Within the area of mechanical stress of the lead caused by the bending, there is a tensiometric tube 27. The tensiometric tube 27 is in the disclosed example assembled to the lead in such a way as to proceed through the lumen of the outer lead conductor 23 being electrically connected to the outer conductor 23 at the point of distal end of the conductor 23 and proximal end of the tube 27. The distal end of the tensiometric tube 27 is electrically connected to the inner lead conductor 21. The tensiometric tube is also isolated by the insulations 24 and 25. The tensiometric tube 27 is electrically connected to the control electronic circuits of an electrotherapy device (not shown) by means of both lead conductors 21 and 23. In the exemplary unipolar configuration the electrode 20 is electrically connected to the electrotherapy circuits of an electrotherapy device by means of the inner lead conductor 21. The bipolar lead should have three lead conductors in order to achieve the proper connection, wherein one conductor should be used only for connection of the tensiometric sensor while one other conductor is common for tensiometric sensor as well as for an electrode, and the third conductor is only used for another electrode.

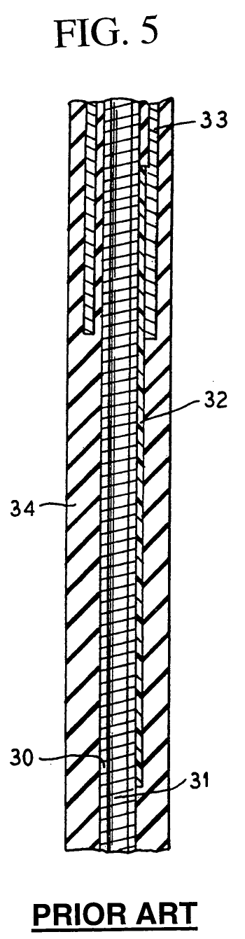

In the embodiment of FIG. 5 there is shown a cross-section of a tensiometric section of a unipolar ventricular lead. The distal end having the active electrode and the proximal end having the connector assembly are not shown. The lead has a lead conductor 30 with a stylet channel 31. The lead conductor 30 connects the active electrode with the corresponding pin on the connector assembly. A section of a tensiometric strip 32, for example made of Kynar® piezoelectric film (Pennwalt Corporation, Valley Forge, Pa.), is mounted tight to the lead conductor 30. Materials such as Kynar®) piezoelectric film have conductive surfaces in order to obtain an electrical connection either by means of either soldering or conductive gluing of electrical conductors on both surfaces. Therefore the lead conductor 30 is tight with the tensiometric strip 32, or conductively glued in such a way as to obtain the electric connection between one surface of the film strip 32 and lead conductor 30. In the disclosed embodiment the lead has helically wounded coaxial lead conductors.

Another surface of the film strip 32 is tight with the outer lead conductor 33 so as to obtain an electric connection between the another conductive surface of the tensiometric film strip 32 and the outer lead conductor 33. In disclosed lead assembly, the electrical connection of the film strip 32 with the connector assembly (not shown) and thus to the control electronic circuits of an electrotherapy device (not shown), is obtained by means of the lead conductors 30 and 33, while the electrical connection of an electrode at the lead tip (not shown) with a corresponding pin on the connector assembly (not shown), and thus to the electrotherapy circuits of an electrotherapy device (not shown), is obtained by means of inner lead conductor 30. The lead body 34 is made of insulation material (either polyurethane or silicone), as it is known in the art, in such a way as to obtain the electrical insulation between the two lead conductors as well as between the lead conductors and the human body tissues and fluid. The disclosed example illustrates the principle of a unipolar tensiometric lead such as a ventricular lead, but the same principle can be applied to the design of a bipolar pacing lead or a multipolar helical-coil lead for an implantable defibrillator. The electrical connection of the tensiometric transducer is obtained in such a way as to use one extra lead conductor for one pole of the transducer and one other lead conductor, which is connected to the one of lead electrodes, for another pole of the transducer. This kind of connection assembly, using one common lead conductor for one pole of the transducer and for one electrode, requires only one additional lead conductor beyond the number of lead conductors normally used in the specific lead type. Of course, a wide variety of different kinds of transducers may be used. Tensiometric tube as well as a tensiometric strip can be made of conductive rubber or any other material, which changes its conductivity because of distension. In such a design the electrotherapy device has to include electronic circuits for measurement of the transducer resistance and analysis of the resistance changes in such a way as to enable detection of various cardiac arrhythmias. Tensiometric tubes and strips can be also made of piezoelectric material, which produces an electric voltage because of distension. In this kind of design the electrotherapy device has to include electronic circuits for measurement and analysis of the transducer signal, thereby enabling the detection and differentiation of various cardiac arrhythmias.

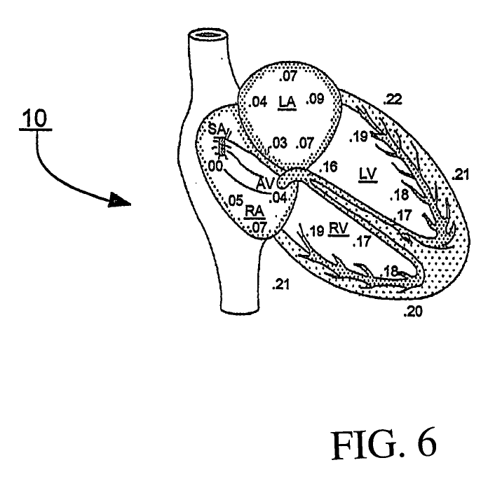

In accordance with an aspect of the present invention, a method and apparatus is provided to restore the normal depolarization-repolarization cardiac cycle sequence of FIG. 6 and the synchrony between the RV, septum, and LV that contributes to adequate cardiac output related to the synchronized electromechanical performance of the RV and LV. The foregoing and other advantages of the invention are realized through delivery of cardiac pacing stimulation to the LV that is timed to occur simultaneously with an intrinsically- or evoked-sensed depolarization in the RV. As a result, the electromechanical performance of RV and LV occur simultaneously or, in the case of LV-only pacing merge into a single “fusion event.” The amount of temporal offset, if any, provided depends on a number of factors. For example, physiologic conduction delay from the A-V node through the His-Purkinje fibers, electrical conduction delay for sensing intracardiac events (from electrodes through threshold sensing circuitry of a medical device), electrical conduction delay for pacing therapy delivery circuitry, ischemic episodes temporarily tempering conduction pathways, myocardial infarction(s) zones, all can deleteriously impact cardiac conduction. Because the conduction status of a patient can vary over time and/or vary based on other factors such as heart rate, autonomic tone and metabolic status, the present invention provides a dynamically controllable bi-ventricular or single-ventricular (e.g., LV-only) pacing modality. For example, for the latter form of pacing, based one or more of several factors, an optimization routine (or sub-routine) can be triggered so that a desired amount of single-chamber fusion-based pacing ensues. Some of the factors include, (i) completion of a pre-set number of cardiac cycles, (ii) pre-set time limit, (iii) loss of capture of the paced ventricle (LV), and/or (iv) physiologic response triggers (e.g., systemic or intracardiac pressure fluctuation, heart rate excursion, metabolic demand increase, decrease in heart wall acceleration, intracardiac electrogram morphology or timing, etc.). The present invention also inherently compensates for the particular implantation sites of the pace/sense electrode pair operatively coupled to the LV chamber.

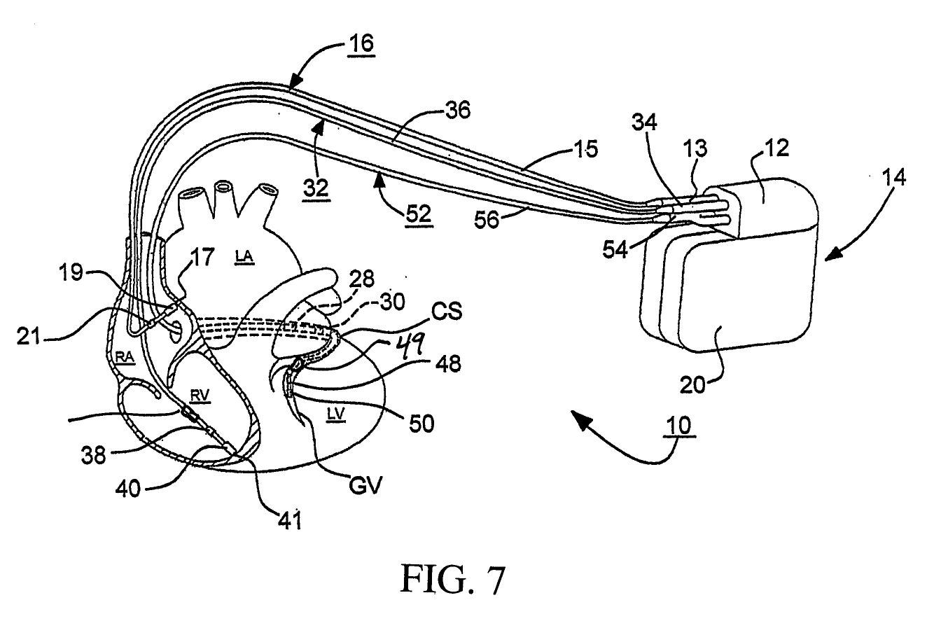

FIG. 7 is a schematic representation of an implanted, triple-chamber cardiac pacemaker comprising a pacemaker IPG 14 and associated leads 16, 32 and 52 in which the present invention may be practiced. The pacemaker IPG 14 is implanted subcutaneously in a patient's body between the skin and the ribs. The three endocardial leads 16,32,52 operatively couple the IPG 14 with the RA, the RV and the LV, respectively. Each lead has at least one electrical conductor and pace/sense electrode, and a remote indifferent can electrode 20 is formed as part of the outer surface of the housing of the IPG 14. As described further below, the pace/sense electrodes and the remote indifferent can electrode 20 (IND_CAN electrode) can be selectively employed to provide a number of unipolar and bipolar pace/sense electrode combinations for pacing and sensing functions, particularly sensing far field signals (e.g. far field R-waves). The depicted positions in or about the right and left heart chambers are also merely exemplary. Moreover other leads and pace/sense electrodes may be used instead of the depicted leads and pace/sense electrodes that are adapted to be placed at electrode sites on or in or relative to the RA, LA, RV and LV. According to the invention, at least one tensiometric mechanical (and/or metabolic) sensor can be deployed independent of, or in tandem with, one or more of the depicted leads.

The depicted bipolar endocardial RA lead 16 is passed through a vein into the RA chamber of the heart 10, and the distal end of the RA lead 16 is attached to the RA wall by an attachment mechanism 17. The bipolar endocardial RA lead 16 is formed with an in-line connector 13 fitting into a bipolar bore of IPG connector block 12 that is coupled to a pair of electrically insulated conductors within lead body 15 and connected with distal tip RA pace/sense electrode 19 and proximal ring RA pace/sense electrode 21. Delivery of atrial pace pulses and sensing of atrial sense events is effected between the distal tip RA pace/sense electrode 19 and proximal ring RA pace/sense electrode 21, wherein the proximal ring RA pace/sense electrode 21 functions as an indifferent electrode (IND_RA). Alternatively, a unipolar endocardial RA lead could be substituted for the depicted bipolar endocardial RA lead 16 and be employed with the IND_CAN electrode 20. Or, one of the distal tip RA pace/sense electrode 19 and proximal ring RA pace/sense electrode 21 can be employed with the IND_CAN electrode 20 for unipolar pacing and/or sensing.

Bipolar, endocardial RV lead 32 is passed through the vein and the RA chamber of the heart 10 and into the RV where its distal ring and tip RV pace/sense electrodes 38 and 40 are fixed in place in the apex by a conventional distal attachment mechanism 41. The RV lead 32 is formed with an in-line connector 34 fitting into a bipolar bore of IPG connector block 12 that is coupled to a pair of electrically insulated conductors within lead body 36 and connected with distal tip RV pace/sense electrode 40 and proximal ring RV pace/sense electrode 38, wherein the proximal ring RV pace/sense electrode 38 functions as an indifferent electrode (IND_RV). Alternatively, a unipolar endocardial RV lead could be substituted for the depicted bipolar endocardial RV lead 32 and be employed with the IND_CAN electrode 20. Alternatively, one of the distal tip RV pace/sense electrode 40 and proximal ring RV pace/sense electrode 38 can be employed with the IND_CAN electrode 20 for unipolar pacing and/or sensing.

In this illustrated embodiment, a bipolar, endocardial coronary sinus (CS) lead 52 having a tensiometric sensor 49 coupled thereto is passed through a vein and the RA chamber of the heart 10, into the coronary sinus and then inferiorly in a branching vessel of the great cardiac vein to extend the proximal and distal LV CS pace/sense electrodes 48 and 50 alongside the LV chamber. The distal end of such a CS lead is advanced through the superior vena cava, the right atrium, the ostium of the coronary sinus, the coronary sinus, and into a coronary vein descending from the coronary sinus, such as the lateral or posteriolateral vein.

In a four chamber or channel embodiment, LV CS lead 52 bears proximal LA CS pace/sense electrodes 48 and 50 positioned along the CS lead body to lie in the larger diameter CS adjacent the LA. Typically, LV CS leads and LA CS leads do not employ any fixation mechanism and instead rely on the close confinement within these vessels to maintain the pace/sense electrode or electrodes at a desired site. The LV CS lead 52 is formed with a multiple conductor lead body 56 coupled at the proximal end connector 54 fitting into a bore of IPG connector block 12. A small diameter lead body 56 is selected in order to lodge the distal LV CS pace/sense electrode 50 deeply in a vein branching inferiorly from the great vein GV.

In this case, the CS lead body 56 would encase four electrically insulated lead conductors extending proximally from the more proximal LA CS pace/sense electrode(s) and terminating in a dual bipolar connector 54. The LV CS lead body would be smaller between the LA CS pace/sense electrodes 28 and 30 and the LV CS pace/sense electrodes 48 and 50. It will be understood that LV CS lead 52 could bear a single LA CS pace/sense electrode 28 and/or a single LV CS pace/sense electrode 50 that are paired with the IND_CAN electrode 20 or the ring electrodes 21 and 38, respectively for pacing and sensing in the LA and LV, respectively.

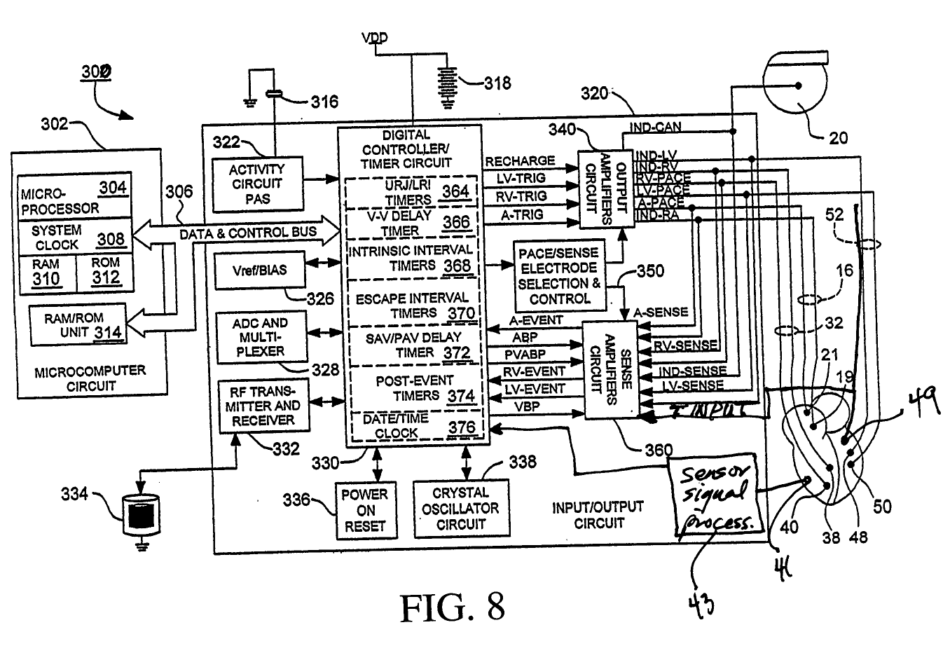

In this regard, FIG. 8 depicts bipolar RA lead 16, bipolar RV lead 32, and bipolar LV CS lead 52 without the LA CS pace/sense electrodes 28 and 30 coupled with an IPG circuit 300 having programmable modes and parameters of a bi-ventricular DDDR type known in the pacing art. The tensiometric sensor 49 id depicted as coupled to the LV in FIG. 8 and operatively coupled to sense amplifier circuit 360 and to other circuitry of circuit 300. In addition, at least one tensiometric, metabolic and/or physiologic sensor 41 is depicted operatively coupled to a portion of RV myocardium and electrically coupled to a sensor signal processing circuit 43. In turn, the sensor signal processing circuit 43 indirectly couples to the timing circuit 330 and via bus 306 to microcomputer circuitry 302. The IPG circuit 300 is illustrated in a functional block diagram divided generally into a microcomputer circuit 302 and a pacing circuit 320. The pacing circuit 320 includes the digital controller/timer circuit 330, the output amplifiers circuit 340, the sense amplifiers circuit 360, the RF telemetry transceiver 322, the activity sensor circuit 322 as well as a number of other circuits and components described below. Of course, in one embodiments of the present invention, the tensiometric sensor 49 can serve in lieu of the sensing function provides by the electrodes 19,21,48,50,38,40 with those electrodes providing only pacing stimulation timed to produce synchronous contractions of the ventricular chambers of the heart. However, in the depicted embodiments both the sensing and pacing circuitry for the electrodes shall be described herein.

Crystal oscillator circuit 338 provides the basic timing clock for the pacing circuit 320, while battery 318 provides power. Power-on-reset circuit 336 responds to initial connection of the circuit to the battery for defining an initial operating condition and similarly, resets the operative state of the device in response to detection of a low battery condition. Reference mode circuit 326 generates stable voltage reference and currents for the analog circuits within the pacing circuit 320, while analog to digital converter ADC and multiplexer circuit 328 digitizes analog signals and voltage to provide real time telemetry if a cardiac signals from sense amplifiers 360, for uplink transmission via RF transmitter and receiver circuit 332. Voltage reference and bias circuit 326, ADC and multiplexer 328, power-on-reset circuit 336 and crystal oscillator circuit 338 may correspond to any of those presently used in current marketed implantable cardiac pacemakers.

If the IPG is programmed to a rate responsive mode, the signals output by one or more physiologic sensor are employed as a rate control parameter (RCP) to derive a physiologic escape interval. For example, the escape interval is adjusted proportionally the patient's activity level developed in the patient activity sensor (PAS) circuit 322 in the depicted, exemplary IPG circuit 300. The patient activity sensor 316 is coupled to the IPG housing and may take the form of a piezoelectric crystal transducer as is well known in the art and its output signal is processed and used as the RCP. Sensor 316 generates electrical signals in response to sensed physical activity that are processed by activity circuit 322 and provided to digital controller/timer circuit 330. Activity circuit 332 and associated sensor 316 may correspond to the circuitry disclosed in U.S. Pat. Nos. 5,052,388 and 4,428,378. Similarly, the present invention may be practiced in conjunction with alternate types of sensors such as oxygenation sensors, pressure sensors, pH sensors and respiration sensors, all well known for use in providing rate responsive pacing capabilities. Alternately, QT time may be used as the rate indicating parameter, in which case no extra sensor is required. Similarly, the present invention may also be practiced in non-rate responsive pacemakers.

Data transmission to and from the external programmer is accomplished by means of the telemetry antenna 334 and an associated RF transmitter and receiver 332, which serves both to demodulate received downlink telemetry and to transmit uplink telemetry. Uplink telemetry capabilities will typically include the ability to transmit stored digital information, e.g. operating modes and parameters, EGM histograms, and other events, as well as real time EGMs of atrial and/or ventricular electrical activity and Marker Channel pulses indicating the occurrence of sensed and paced depolarizations in the atrium and ventricle, as are well known in the pacing art.

Microcomputer 302 contains a microprocessor 304 and associated system clock 308 and on-processor RAM and ROM chips 310 and 312, respectively. In addition, microcomputer circuit 302 includes a separate RAM/ROM chip 314 to provide additional memory capacity. Microprocessor 304 normally operates in a reduced power consumption mode and is interrupt driven. Microprocessor 304 is awakened in response to defined interrupt events, which may include A-TRIG, RV-TRIG, LV-TRIG signals generated by timers in digital timer/controller circuit 330 and A-EVENT, RV-EVENT, and LV-EVENT signals generated by sense amplifiers circuit 360, among others, in response to either (or both) the electrodes 19,21,48,50,38,40 and the tensiometric sensor 49. The specific values of the intervals and delays timed out by digital controller/timer circuit 330 are controlled by the microcomputer circuit 302 by means of data and control bus 306 from programmed-in parameter values and operating modes. In addition, if programmed to operate as a rate responsive pacemaker, a timed interrupt, e.g., every cycle or every two seconds, may be provided in order to allow the microprocessor to analyze the activity sensor data and update the basic A-A, V-A, or V-V escape interval, as applicable. In addition, the microprocessor 304 may also serve to define variable AV delays and the uni-ventricular, pre-excitation pacing delay intervals (A-LVp) from the activity sensor data, metabolic sensor(s) 41 and/or mechanical sensor(s) 49.

In one embodiment of the invention, microprocessor 304 is a custom microprocessor adapted to access and execute instructions stored in RAM/ROM unit 314 in a conventional manner. It is contemplated, however, that other implementations may be suitable to practice the present invention. For example, an off-the-shelf, commercially available microprocessor or microcontroller, or custom application-specific, hardwired logic, or state-machine type circuit may perform the functions of microprocessor 304.

Digital controller/timer circuit 330 operates under the general control of the microcomputer 302 to control timing and other functions within the pacing circuit 320 and includes a set of timing and associated logic circuits of which certain ones pertinent to the present invention are depicted. The depicted timing circuits include URI/LRI timers 364, V-V delay timer 366, intrinsic interval timers 368 for timing elapsed V-EVENT to V-EVENT intervals or V-EVENT to A-EVENT intervals or the V-V conduction interval, escape interval timers 370 for timing A-A, V-A, and/or V-V pacing escape intervals, an AV delay interval timer 372 for timing the A-LVp delay (or A-RVp delay) from a preceding A-EVENT (optionally as a sensed-AV, or “SAV,” interval) or A-TRIG (optionally as a paced-AV, or “PAV,” interval), a post-ventricular timer 374 for timing post-ventricular time periods, and a date/time clock 376.

In the present invention, the AV delay interval timer 372 is loaded with an appropriate delay interval for the ventricular chambers (i.e., an A-RVp delay and/or an A-LVP delay as determined by described and/or depicted elsewhere herein) to time-out starting from a preceding A-PACE or A-EVENT. The interval timer 372 times the interval, and is based on one or more prior cardiac cycles (or from a data set empirically derived for a given patient) and does not necessarily depend on sensing of a depolarization in the other ventricle (e.g., RV) during fusion-based pacing therapy delivery according to one form of the invention.

The post-event timers 374 time out the post-ventricular time periods following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 302. The post-ventricular time periods include the PVARP, a post-atrial ventricular blanking period (PAVBP), a ventricular blanking period (VBP), and a ventricular refractory period (VRP). In the event that any of the electrode pairs are being employed to sense electrical activity of the heart, the post-atrial time periods typically include blanking periods. That is, an atrial refractory period (ARP) during which an A-EVENT is ignored for the purpose of resetting any AV delay, and an atrial blanking period (ABP) during which atrial sensing is disabled. It should be noted that the starting of the post-atrial time periods and the AV delays can be commenced substantially simultaneously with the start or end of each A-EVENT or A-TRIG or, in the latter case, upon the end of the A-PACE, which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 304 also optionally calculates AV delays, post-ventricular time periods, and post-atrial time periods that vary with the sensor based escape interval established in response to the RCP(s) and/or with the intrinsic atrial rate.

The output amplifiers circuit 340 contains a RA pace pulse generator (and a LA pace pulse generator if LA pacing is provided), a RV pace pulse generator, and/or a LV pace pulse generator or corresponding to any of those presently employed in commercially marketed cardiac pacemakers providing atrial and ventricular pacing.

In order to trigger generation of an RV-PACE and/or LV-PACE pulse, the temporal separation of the output signal of tensiometric sensor 49 related to sensed contractions of the LV and RV (if any) can be utilized as the primary or exclusive source for the timed delivery of atrial and/or ventricular pacing stimuli. Alternatively, the digital controller/timer circuit 330 generates the RV-TRIG signal at the time-out of the A-RVp delay (in the case of RV pacing) or the LV-TRIG at the time-out of the A-LVP delay (in the case of LV pacing) provided by AV delay interval timer 372 (or the V-V delay timer 366). Similarly, digital controller/timer circuit 330 generates an RA-TRIG signal that triggers output of an RA-PACE pulse (or an LA-TRIG signal that triggers output of an LA-PACE pulse, if provided) at the end of the V-A escape interval timed by escape interval timers 370.

The output amplifiers circuit 340 includes switching circuits for coupling selected pace electrode pairs from among the lead conductors and the IND_CAN electrode 20 to the RA pace pulse generator (and LA pace pulse generator if provided), RV pace pulse generator and LV pace pulse generator. Pace/sense electrode pair selection and control circuit 350 selects lead conductors and associated pace electrode pairs to be coupled with the atrial and ventricular output amplifiers within output amplifier circuit 340 for accomplishing RA, LA, RV and LV pacing.

The sense amplifier circuit 360 contains sense amplifiers corresponding to any of those presently employed in contemporary cardiac pacemakers for atrial and ventricular pacing and sensing. As noted in the above-referenced, commonly assigned, '324 patent, it has been common in the prior art to use very high impedance P-wave and R-wave sense amplifiers to amplify the voltage difference signal which is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 330 controls sensitivity settings of the atrial and ventricular sense amplifiers 360.

Unlike the tension metric sensor(s) 49 employed according to the invention, the sense amplifiers are uncoupled from the sense electrodes during the blanking periods before, during, and after delivery of a pace pulse to any of the pace electrodes of the pacing system to avoid saturation of the sense amplifiers. The sense amplifiers circuit 360 includes blanking circuits for uncoupling the selected pairs of the lead conductors and the IND_CAN electrode 20 from the inputs of the RA sense amplifier (and LA sense amplifier if provided), RV sense amplifier and LV sense amplifier during the ABP, PVABP and VBP. The sense amplifier circuit 360 also includes switching circuits for coupling selected sense electrode lead conductors and the IND_CAN electrode 20 to the RA sense amplifier (and LA sense amplifier if provided), RV sense amplifier and LV sense amplifier. Again, sense electrode selection and control circuit 350 selects conductors and associated sense electrode pairs to be coupled with the atrial and ventricular sense amplifiers within the output amplifiers circuit 340 and sense amplifiers circuit 360 for accomplishing RA, LA, RV and LV sensing along desired unipolar and bipolar sensing vectors.

Right atrial depolarizations or P-waves in the RA-SENSE signal that are sensed by either (or both) the tensiometric sensor 49 and the RA sense amplifier result in a RA-EVENT signal that is communicated to the digital controller/timer circuit 330. Similarly, left atrial depolarizations or P-waves in the LA-SENSE signal that are sensed by either or both the tensiometric sensor 49 and the LA sense amplifier, if provided, result in a LA-EVENT signal that is communicated to the digital controller/timer circuit 330. Ventricular depolarizations or R-waves in the RV-SENSE signal are sensed by either (or both) the tensiometric sensor 49 and a ventricular sense amplifier result in an RV-EVENT signal that is communicated to the digital controller/timer circuit 330. Similarly, ventricular depolarizations or R-waves in the LV-SENSE signal are sensed by a ventricular sense amplifier result in an LV-EVENT signal that is communicated to the digital controller/timer circuit 330. Unlike the output signals from tensiometric sensor 49, the RV-EVENT, LV-EVENT, and RA-EVENT, LA-SENSE signals may be refractory or non-refractory, and can inadvertently be triggered by electrical noise signals or aberrantly conducted depolarization waves rather than true R-waves or P-waves. Because the tensiometric sensor 49 monitors only mechanical contractions it is not deleteriously influenced by refractory or non-refractory status of the myocardium, or by mis-conducted P- or R-waves. Thus, as can be appreciated by those of skill in the art, the present invention provides advantages alone or in combination with traditional electrical cardiac activity sensing circuitry in the context of cyclical pacing, sensing and detecting cardiac arrthymias, among other advantages.

In addition to the foregoing, it will be understood that specifically described structures, functions and operations set forth in the above-referenced patents can be practiced in conjunction with the present invention, but they are not essential to its practice. It is therefore to be understood, that within the scope of the appended claims, the invention may be practiced otherwise than as specifically described without actually departing from the spirit and scope of the present invention.

1. A system for continuously sensing mechanical activity of a heart and adjusting a pacing therapy based on the sensed mechanical activity, comprising:
a processor-based electronic cardiac pacing engine; and
a single mechanical sensor adapted to detect cardiac contractions of at least a left atrial chamber, a left ventricular chamber, and a right ventricular chamber and provide an output signal corresponding to said detected cardiac contractions to the processor-based electronic cardiac pacing engine.

2. A system according to claim 1, wherein said single mechanical sensor is adapted to be coupled at least one of the following:
a portion of a coronary sinus ostium,
a portion of a coronary sinus,
a portion of a cardiac vein.

3. A system according to claim 1, further comprising an additional mechanical sensor adapted to mechanically couple to a discrete portion of the right ventricular chamber

4. A system according to claim 1, wherein the single mechanical sensor comprises one of a tensiometric-type sensor and an accelerometer sensor.

5. A system according to claim 4, wherein said accelerometer sensor comprises one of a single axis accelerometer and a multiple axis accelerometer.

6. A system according to claim 4, wherein the tensiometric-type sensor further comprises a transvenous delivery mechanism coupled to said tensiometric-type sensor.

7. A system according to claim 6, wherein said transvenous delivery mechanism comprises one of: a stylet, a single lumen delivery catheter, a guidewire.

8. A system according to claim 3, wherein the additional mechanical sensor comprises one of a tensiometric-type sensor and an accelerometer sensor.

9. A system according to claim 8, wherein said accelerometer sensor comprises one of a single axis accelerometer and a multiple axis accelerometer.

10. A system according to claim 8, wherein the tensiometric-type sensor further comprises a transvenous delivery mechanism coupled to said tension-metric sensor.

11. A system according to claim 10, wherein said transvenous delivery mechanism comprises one of: a stylet, a single lumen delivery catheter, a guidewire.

12. A system according to claim 1, wherein the processor-based electronic cardiac pacing engine comprises an implantable pulse generator.

13. A system according to claim 1, wherein the processor-based electronic cardiac pacing engine comprises an implantable cardioverter-defibrillator.

14. A system according to claim 1, wherein the processor-based electronic cardiac pacing engine further comprises a programmable medium for executing computer readable instructions.

15. A system according to claim 14, wherein the computer readable medium includes instructions for delivering one of: a bradycardia pacing modality, a tachycardia pacing modality, a cardiac resynchronization therapy modality, a single-chamber pacing modality.

16. A system according to claim 14, wherein the computer readable medium includes instructions for delivering a cardiac resynchronization therapy modality.

17. A system according to claim 1, wherein the processor-based electronic cardiac pacing engine comprises an external pulse generator.

18. A method of delivering cardiac resynchronization therapy with a system for continuously mechanically sensing contractions of a heart chamber, said system comprising a processor-based electronic component operatively electrically coupled to a mechanical sensor adapted to be mechanically coupled to a left ventricle so that said sensor provides an output signal for each one of atrial contractions, left ventricular contractions, and right ventricular contractions and wherein said stylet is in electrical communication with the processor-based electronic component, said method comprising:
sensing an atrial contraction and providing a temporal output signal related thereto;
sensing at least one ventricular contraction and if the at least one ventricular contraction comprises a single discrete ventricular contraction event, then:
continuing to deliver a cardiac resynchronization therapy without modifying any pacing therapy delay intervals; and if the at least one ventricular contraction comprises two discrete ventricular contraction events, then:
electrically stimulating a first ventricular chamber at an interval of time prior to or later than a second ventricular chamber;
detecting a contraction event of the first ventricular chamber relative to the second ventricular chamber;
modifying the interval of time until the at least one ventricular contraction comprises the single discrete ventricular contraction event; and
continuing to deliver the cardiac resynchronization therapy without further modifying the interval of time.

19. A computer-readable medium for delivering cardiac resynchronization therapy with a system for continuously mechanically sensing contractions of a heart chamber, said system comprising a processor-based electronic component operatively electrically coupled to a mechanical sensor adapted to be mechanically coupled to a left ventricle so that said sensor provides an output signal for each one of atrial contractions, left ventricular contractions, and right ventricular contractions and wherein said stylet is in electrical communication with the processor-based electronic component, said method comprising:
instructions for sensing an atrial contraction and providing a temporal output signal related thereto;
instructions for sensing at least one ventricular contraction and if the at least one ventricular contraction comprises a single discrete ventricular contraction event, then:
instructions for continuing to deliver a cardiac resynchronization therapy without modifying any pacing therapy delay intervals; and if the at least one ventricular contraction comprises two discrete ventricular contraction events, then:
instructions for electrically stimulating a first ventricular chamber at an interval of time prior to or later than a second ventricular chamber;
instructions for detecting a contraction event of the first ventricular chamber relative to the second ventricular chamber;
instructions for modifying the interval of time until the at least one ventricular contraction comprises the single discrete ventricular contraction event; and
instructions for continuing to deliver the cardiac resynchronization therapy without further implementing instructions for modifying the interval of time.

20. A medium according to claim 5, further comprising instructions for detecting a contraction of an atrial chamber with the single tensiometric-sensing stylet disposed in or about the portion of the coronary sinus of the patient.
